(19) United States
(12) Patent Application Publication
(10) Pub. No.: US 2009/0022779 A1
(43) Pub. Date: Jan. 22, 2009

(54) Phenytoin Formulations, and Uses Thereof in Wound Healing

(75) Inventors: John Kelly, Dublin (IE); Cormac Kennedy, Co. Limerick (IE); Clare Meaney, Dublin (IE)

Correspondence Address: WOLF GREENFIELD & SACKS, P.C., 600 ATLANTIC AVENUE, BOSTON, MA, 02210-2206, US

(73) Assignee: Royal College of Surgeons in Ireland, Dublin 2 (IE)

(21) Appl. No.: 11/659,051

(22) PCT Filed: Aug. 2, 2005
(86) PCT No.: PCT/EP05/08344

§ 371 Date: Sep. 11, 2008

Related U.S. Application Data
(60) Provisional application No. 60/598,479, filed on Aug. 3, 2004.

(51) Int. Cl. A61L 15/00; A61P 17/02

(52) U.S. Cl. 424/445; 424/447; 424/486

(57) ABSTRACT

A formulation of phenyloin suitable for topical application to a wound comprises a reservoir of phenyloin entrapped within a stabilising matrix, and an amount of dissolved phenyloin, wherein the dissolved phenyloin is in chemical equilibrium with the phenyloin entrapped within the stabilised matrix. The stabilised matrix may comprise a gel matrix, especially a gel matrix in which the polymer of the gel forms ion-pairs with the phenyloin. Also described are methods of treating a wound in diabetic and non-diabetic patients using a formulation according to the invention.

### TECHNICAL FIELD

This invention relates to formulations of phenyloin suitable for topical application to a wound. In particular, the invention relates to gel-based formulations of phenyloin. The invention also relates to methods for the topical treatment of wounds.

### BACKGROUND ART

Phenyloin (diphenylhydantoin, 5,5-diphenylimidazolidine-2,4-dione) has well-established clinical use as an anticonvulsant and is used as an anti epileptic agent. It also has cardiac antiarrhythmic properties. Phenyloin can be administered orally or (as the sodium salt) by injection.

Despite the usefulness of phenyloin for treating seizures, it has a number of side effects, which are well described in the literature (see for example Goodman & Gilman's The Pharmacological Basis of Therapeutics, McGraw Hill). These side effects include gastrointestinal disturbances, nervous system toxicity, various central nervous system effects, various endocrine effects, hirsutism, cardiac arrhythmias, liver damage, hypersensitivity reactions and interactions with other medications.

One frequently observed side effect is tenderness and enlargement of the tissues of the gums (gingival hyperplasia). Phenyloin is the most common cause of drug-induced gingival enlargement. However it is also associated with use of cyclosporin and calcium channel blockers (Desai P & Silver J G, J Can Dent Assoc. 64(4): 263-8, 1998).

At least 20% and perhaps up to about 50% of patients treated with phenyloin develop gingival enlargement as a side effect. The incidence may increase as the plasma concentration of phenyloin increases (Perlik F et al. Ther Drug Monit. 17(5) 445-8 1995). This stimulatory effect on tissue growth has prompted the evaluation of phenyloin in wound healing.

The literature contains results of clinical investigations and case reports of treatments in which the application of phenyloin had beneficial properties in wound healing in various types of wounds. These include experimental models of incised wounds (DaCosta M L, Surgery 123(3) 287-93 1998) and fractures (Frymoyer J W, J Trauma 16(5) 368-70, 1976) and clinical studies in traumatic wounds (Modaghegh S, et al. Int J. Dermatol. 28(5) 347-50 1989), ulcers (Anstead G M, et al. Ann Pharmacother. 30(7-8) 768-75 1996; Bansal N K, Int J. Dermatol. 32(3): 210-3, 1993) and skin grafts (Yadav J K, Burns, 19(4): 306-10, 1993).

The mechanism of action of phenyloin in wound healing is unknown but is likely to be related to the otherwise adverse effect of gingival tissue enlargement rather than to its anticonvulsant properties. A number of studies have shown that phenyloin increases the production of various cytokines and growth factors, including interleukin-1b (Modeer T, et al. Life Sci. 44(1) 35-401 1989), keratinocyte growth factor (Das S J and Olsen I, Biochem Biophys Res Commun. 13 282(4) 875-81 2001), platelet-derived growth factor (Iacopino A M, et al. J. Periodontol. 68(1) 73-83 1997) and basic fibroblast growth factor (Sasaki T and Maita E, J Clin Periodontol. 25(1) 42-7 1998). Phenyloin altered the expression of a number of genes in dermal fibroblasts. (Swamy S M K, et al. Biochem Biophys Res Commun. 314 661-666 2004).

The above investigations employed phenyloin or a salt contained in or added to a biological medium in the case of in vitro studies, applied topically as a solution, suspension or powder for in vivo or clinical studies, or in some cases administered by injection, for example in studies with bone fractures. The liquid or powder applications are mostly derived from the commercially available injections or capsules. They do not teach how phenyloin may be used in a suitable pharmaceutical composition that can be conveniently and accurately used in the treatment of wounds in patients who would benefit from such treatment. Lasker (U.S. Pat. No. 5,571,521) describes the use of a silver ammonium phenyloin complex, together with phenyloin, in the treatment of wounds and particularly as biocides for the treatment of infections. However no description is given of the pharmaceutical compositions necessary to achieve these ends.

Phenyloin is only slightly soluble in water. Phenyloin sodium salt is soluble in water but only remains in solution in significant amounts at alkaline pH, typically at pH values of approximately 12. Exposure of wound tissue to this pH is not desirable. At lower values of pH there is an increased proportion of undissolved phenyloin.

Given the acknowledged efficacy of phenyloin in promoting the healing of wounds, there is a clear need in the art for methods whereby this property may be provided in a convenient, efficacious, stable and reproducible manner for use by patients and their carers. Preferably there should be means to ensure a suitable residence time and a method of ensuring uptake of the agent by the cells and tissues of the wound.

### STATEMENTS OF INVENTION

According to the invention, there is provided a formulation of phenyloin suitable for topical application to a wound comprising a reservoir of phenyloin entrapped within a stabilising matrix, and an amount of dissolved phenyloin, wherein the dissolved phenyloin is in chemical equilibrium with the phenyloin entrapped within the stabilised matrix.

In this specification, the term “wound”, unless otherwise indicated, is intended to mean both chronic wounds such as those associated with long-term illness such as diabetes and those associated with immobility such as decubitus ulcers, and acute wounds such as those of a traumatic origin.

In the formulations of the invention, the stabilising matrix functions to partition the phenyloin in the formulation into a component that is dissolved in the formulation, generally an aqueous component of the formulation, and which is free to perform a therapeutic function, and a component that acts as a reservoir of phenyloin that, in use, continually replenishes the dissolved phenyloin as the dissolved phenyloin is used in the wound being treated. Thus, the formulation provides a controlled and sustained release of phenyloin which, in one embodiment, utilises the intrinsic low solubility of phenyloin to achieve these favourable release characteristics.

In a preferred embodiment, the stabilised matrix comprises a gel, wherein the dissolved phenyloin is located in an aqueous component of the gel. In such cases, the polymer matrix of the gel functions to physically or electrochemically entrap the phenyloin. Typically, the gel is formed by a gelling agent selected from the group comprising: alginic acid; alginate derivatives; chitosan; chitosan derivatives; methylcellulose; methylcellulose derivatives; microcrystalline cellulose; carboxymethylcellulose salts; hydropropylmethyl cellulose; polyvinylpyrrolidone; tragacanth; and carrageenan. Suitably, the gel comprises an acidic polymer.

In a particularly preferred embodiment of the invention, the gel is formed by a gelling agent that is capable of forming an ion pair with the phenyloin. Suitable gelling agents include carbomers (i.e. CARBOPOL) and alginates. Such gelling agents include carboxylic acid groups that ion pair with basic groups on the phenyloin molecule, thereby increasing the stability of the reservoir of phenyloin.

Generally, the gel-forming gelling agent is present in the formulation in an amount of from 0.5% to 10.0% (w/w), suitably from 0.5% to 5%, preferably from 0.5% to 3%, more preferably from 0.5% to 2%, and ideally at about 1% (w/w).

In one embodiment of the invention, the stabilised matrix is formed by a complexing agent which functions to physically or chemically entrap the phenyloin in the reservoir. Suitable stabilising agent may be selected from the group comprising: cyclodextrins; buffer salts; amino acids; small peptides; polyarginines; polyglycines; polylysines; and glutamic acid. A particularly suitable complexing agent is hydroxypropyl-β-cyclodextrin. Typically, the formulation will include a complexing agent in an amount of from 1% to 50% (w/w), preferably from 20% to 40% (w/w), and more preferably from 25% to 35% (w/w). In one preferred embodiment of the invention, the formulation will include a complexing agent along with one or more of gelling agents. In this regard, formulations that include a carbomer and a cyclodextrin have been found to be particularly advantageous.

In one embodiment of the invention, the stabilised matrix is formed by an oil-in-water (o/w) or a water-in-oil (w/o) emulsion base, in which the dissolved phenyloin is located in a water phase and the phenyloin reservoir is located in the oil phase. Ideally, the stabilised matrix is an oil-in-water emulsion base. In such cases, phenyloin reservoir will be entrapped within the oil droplets dispersed within the water, or aqueous, phase but will remain in equilibrium with the dissolved phenyloin in the continuous phase. Such formulations will generally include means for stabilising the emulsion base. Stabilisers for emulsions, and particularly aqueous emulsions in which the continuous phase is substantially aqueous, will be well known to those skilled in the art.

In one embodiment of the invention, in which the stabilised matrix is formed by an emulsion base, the formulation may additionally include components which allow the formulation to foam on being dispensed from a suitable dispenser with a suitable propellant. Components suitable for foam formation will be well known to those skilled in the art and may include solvents, co-solvents, additional lipophilic components, and the like. In such cases, the foam will include a gas phase, an aqueous phase in which the dissolved phenyloin exists, and a dispersed lipophilic phase that contains the reservoir of phenyloin. The reservoir of phenyloin will be in equilibrium with the dissolved phenyloin in the aqueous phase. Accordingly, the invention also relates to a foam-forming formulation of the invention, in which the stabilised matrix is formed by an emulsion base, typically an oil-in-water emulsion base in which the phenyloin reservoir is located in the dispersed oil phase, and wherein the formulation includes components which allow the formulation to foam on being dispensed from a suitable dispenser with a suitable propellant. The invention also relates to a dispenser suitable for dispensing a foam, in combination with a foam-forming formulation of the invention and a suitable propellant, wherein the formulation and the propellant are contained within the dispenser, typically under pressure.

In one embodiment, the phenyloin is present in the formulation at an amount of from 0.5% to 10.0%, preferably from 1.0% to 7.0%, and more preferably from 2.0% to 6.0% (w/w). Ideally, the phenyloin is present at an amount of from 3.0% to 5.0% (w/w). Suitably, the phenyloin comprises phenyloin acid, a phenyloin salt, a derivative of phenyloin, or a mixture thereof. In one embodiment, the derivative of phenyloin includes pro-drugs such as that sold under the Trade Name FOSPHENYTOIN. Ideally, the phenyloin salt consists of sodium phenyloin.

Typically, the formulation of the invention has a pH of from 4 to 12, more preferably from 7 to 10.

In one embodiment, the formulation of the invention includes one or more therapeutic molecules for the treatment of wounds, such as, for example, anti-infective agents selected from the group comprising antibiotics, antifungal agents, and zinc salts.

In one particularly suitable embodiment of the invention, the formulation comprises:
from 0.5% to 10.0% phenyloin salt;
from 0.5% to 5.0% gelling agent;
an aqueous base; and optionally
an amount of alkali or acid or buffer salts sufficient to adjust the pH of the formulation to between 7 and 10.

Typically, the gelling agent comprises a polymer that is capable of forming an ion-pair with the phenyloin. Examples of such gelling agents include carbomers (i.e. CARBOPOL) and alginates.

Suitably, the formulation additionally includes a complexing agent for the phenyloin. A preferred complexing agent is a cyclodextrin.

Ideally, the gelling agent comprises from 0.5% to 1.5% of the formulation (w/w). Generally, the cyclodextrin may be present at from 20% to 40% (w/w).

The invention also relates to a solid support forming part of a bandage or a dressing for wounds, wherein the solid support carries a formulation according to the invention. Typically, the solid support comprises a mesh formed of woven or non-woven material. The invention also relates to a bandage or dressing for wounds comprising a solid support according to the invention.

The invention also relates to a formulation of the invention for use as a medicament.

The invention also relates to a formulation of the invention for use in the topical treatment of wounds.

A formulation according to the invention for use in the topical treatment of wounds in diabetic patients.

The invention also relates to a method of treating a wound in an individual comprising a step of administering a formulation according to the invention topically to the wound.

The invention also relates to a method of treating a wound in a diabetic patient comprising a step of administering a formulation according to the invention topically to the wound.

The invention also provides a means for treating a wound in individuals suffering from burn trauma. In one embodiment, the invention provides the combination of a dispenser adapted for delivering a liquid in the form of an aerosol, mist, spray or the like, a phenyloin-containing formulation contained within the dispenser along with a suitable propellant for the formulation, the phenyloin formulation comprising phenyloin and a solvent for the phenyloin. In this regard, the invention also relates to the use of a liquid phenyloin-containing formulation suitable for topical application in the form of a spray, mist or aerosol, in the manufacture of a medicament for the treatment of wounds located in burnt tissue, wherein the liquid phenyloin-containing formulation comprises phenyloin, and a solvent for the phenyloin The invention also relates to a method of topically treating wounds located in or around burnt tissue, comprising the step of applying a liquid phenyloin-containing formulation onto the wound in the form of a mist, spray or aerosol. Typically, the solvent is substantially aqueous. In one embodiment, the phenyloin-containing formulation includes a complexing agent for the phenyloin. Suitable complexing agents are hereinbefore described. The use of phenyloin for treating wounds in victims of burn trauma has the added benefit of having an anaesthetic effect on the wound due to the sodium channel blocking activity of the phenyloin.

The present invention takes the form of formulations of phenyloin in which there is an equilibrium among dissolved and suspended phenyloin, phenyloin sodium and free phenyloin. The relative proportions of all of these depend upon the pH, the original concentrations of phenyloin and phenyloin sodium and any solubilising excipients in the formulation. These multi component systems contain a slowly releasing reservoir of phenyloin in equilibrium with dissolved phenyloin in the formulation and further in equilibrium with phenyloin in the physiological environment of the wound, including the physiological fluid surrounding the cells and tissue of the wound, the cell membrane and the intracellular environment. The present invention encompasses topical formulations of phenyloin having pH values in the range 4-12, but particularly having pH values in the range 7-10, which contain phenyloin free substance or the sodium salt or a combination of these, such that there is an equilibrium between dissolved and suspended phenyloin.

The invention is a topical, extended release formulation of phenyloin in which there is an equilibrium among a phenyloin reservoir and free phenyloin in the formulation and in the physiological environment of the wound. The composition of the reservoir influences the amount of phenyloin available to the wound and the time course of the exposure of the wound to the therapeutic effect of phenyloin.

The components of the formulation are as follows
1. A reservoir of phenyloin. This may take the form of a suspension of phenyloin or a salt of phenyloin and may be a reservoir in which phenyloin or a salt is included in a matrix in a complexed or intercalated form such as that formed by mixing with a cyclodextrin.
2. Free phenyloin or a salt of phenyloin dissolved in the formulation. This is in equilibrium with the phenyloin contained in the reservoir. The relative amounts of phenyloin in each component will be influenced by the composition of the formulation, including the pH and the presence of excipients that influence the solubility of phenyloin.

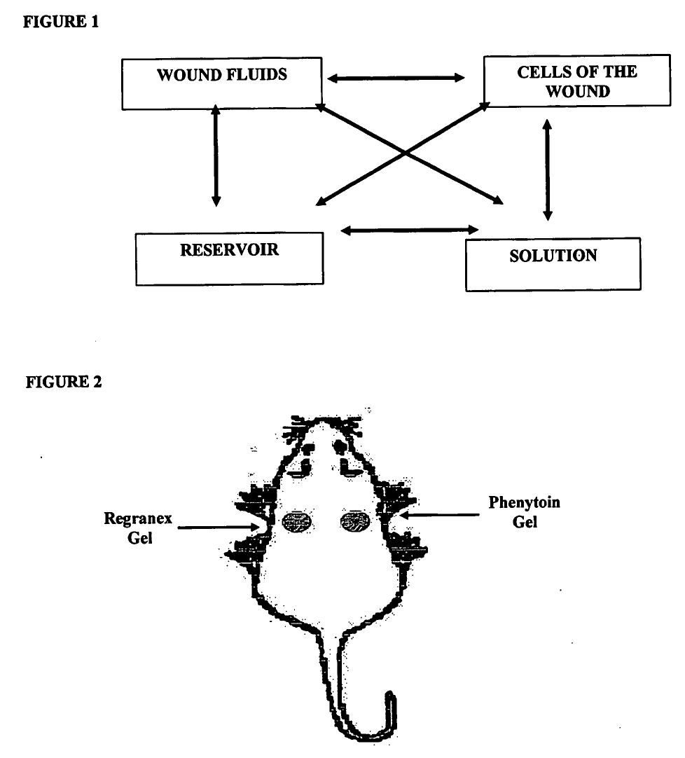

Upon application to the wound a further set of equilibrium conditions are established among phenyloin or its salt in the reservoir, dissolved in the formulation, suspended or dissolved in the physiological fluid surrounding the cells and tissues of the wound and phenyloin in the cell membranes and the intracellular environment of the wound. A diagram illustrating the set of equilibrium conditions is shown in FIG. 1.

The operation of the invention following application to the wound is illustrated in the above figure. Phenyloin is present in all of the compartments and the equilibrium conditions can be varied by adjusting the various constituents of the formulation. For example the ultimate duration of action can be made dependent upon the amount of phenyloin in the reservoir. The amount of phenyloin made available to the cells and tissue of the wound can be made dependent upon the pH of the formulation or the nature and amount of excipients.

While the application of this invention to phenyloin and phenyloin sodium and to suspensions of phenyloin and cyclodextrin complexes is described above, its applicability to other salts and complexes of phenyloin and its analogues may readily be appreciated and the incorporation of additional therapeutic molecules for the treatment of wounds is envisaged. Notable among these is the use of antimicrobial substances, for example anti-infective agents such as antibiotics, antifungals and zinc salts.

The topical formulations of the present invention can take various forms. For example, creams (emulsions), lotions, gels, and aqueous liquids are all contemplated. Also contemplated are formulations applied by spraying, such as mist, aerosol or foam spraying. A difference between these forms is their physical appearance and viscosity, which can be governed by the presence and amount of emulsifiers and viscosity adjusters present in the formulation. Gels provide a particularly useful form of the invention. They are semisolid and liquid rich and form a suitable compatible formulation for application to wounds. They can be prepared with a range of viscosities. Their formulations may contain solvents, emulsifiers, moisturizers, emollients, preservatives and other active ingredients that increase or enhance the efficacy of the final product. The presence of solvents can contribute to the modulation of release rates. The invention contemplates topical formulations designed for controlled-release of phenyloin to a wound surface.

As mentioned above, gels are a particularly useful form of the invention. It can be readily understood that the gel may be presented in various forms and viscosities that range from the essentially liquid to relatively solid. It may be squeezed from a deformable multiple application container, from suitable unit dose containers or applied as sheets or be prepared on solid matrices, such as dressings. The above are examples and are not intended to form an exhaustive list but serve to illustrate the methods.

Surprisingly we have discovered that the use of a Carbomer has a counter-ion effect that results in a stable, smooth dispersion of phenyloin at pH values close to physiological, in contrast to simple aqueous preparations. Therefore the use of a Carbomer as a gelling agent is particularly advantageous and these preparations constitute a preferential form of the present invention.

Surprisingly also we have discovered that the invention displays particular utility in the treatment of wounds in diabetic conditions and the use of the invention in these wounds is a preferential but not exclusive indication.

Another useful form of the invention is a liquid suitable for application to a wound as a lotion or as a spray.

By altering the various components of the formulation, products of differing durations of action can be produced and extended release products offering convenience to patients and their carers are envisaged. Various frequencies of administration can be envisaged from say once daily to once weekly or indeed as a single administration. These are examples and do not seek to confine the frequency or total number of administrations.

### DETAILED DESCRIPTION OF THE INVENTION

The invention will be more clearly understood from the following description of some embodiments thereof, given by way of example only, with reference to the accompanying figures and graphs.

### Example 1

### Extended-Release Phenyloin Gel

Carbomer 974 PNF 1 g is added to approximately 80 g deionised water. Following complete addition of Carbomer, the preparation is mixed for 30 minutes. Phenyloin sodium 5 g is gradually added while mixing. The gel thickens. The gel is made up to 100 g with deionised water, with mixing. The pH is adjusted to 7.4 with sodium hydroxide.

### Example 2

### Extended-Release Phenyloin Gel

Hydroxypropyl-β-Cyclodextrin 30 g is dissolved in approximately 60 g deionised water with stirring. Phenyloin sodium 1 g is added slowly to form a suspension. Carbomer 974 PNF 1 g is gradually added with mixing at 1500 rpm. The gel is made up to 100 g with deionised water, with mixing. The pH is adjusted to 7.4 with sodium hydroxide.

### Example 3

### Efficacy Model

A rat dorsal wound model as described by DaCosta et al. (Surgery 123(3) 287-93 1998) was employed. Wounds were treated by insertion of aliquots (0.2 g) of either the phenyloin-containing gel described in Example 2 above, or a gel manufactured without phenyloin. Ten days afterwards, the integrity of the wounds were examined and compared. The beneficial effects of the invention were manifested by an increase in wound tensile strength of approximately 30%, when compared with the inactive treatment, accompanied by increased amounts of hydroxyproline, which is a marker of collagen deposition.

### Example 4

### Efficacy Model—Diabetes

A full-thickness excisional wound were studied in rats rendered diabetic by the administration of streptozotocin. Wounds were treated by application of aliquots of either the phenyloin-containing gel described in Example 2 above, or a gel manufactured without phenyloin. The improvement in wound healing in the diabetic animal (demonstrated by a decrease in wound area) was at least 50% greater than placebo-treated animals at day 9 and day 12.

### Example 5

### Comparison with Commercially Available Product

Male Sprague-Dawley rats were rendered diabetic by a single intraperitoneal injection of streptozotocin (STZ). Serum glucose was measured from a sample of venous blood taken from the tail vein of each diabetic rat one week after STZ administration using a glucometer and dextroslide. In this excisonal wound model, two circular wounds with a diameter of 20 mm were created at equal distance from the midpoint and symmetrically on the dorsum of the rat below the inferior edge of the scapula, as shown in FIG. 2. Treatments were topically applied once daily to the wounds. The area of the wound was traced onto a transparent sheet every third day and the tracing scanned. Wound area was calculated with a non-rectangular area analysis. The serum concentration of phenyloin was measured by sampling at the end of the experimental period. Blood was draw by intra-cardiac puncture biochemically analysed for phenyloin. All animals were housed in a licensed biomedical research facility under normal laboratory conditions and the study was conducted with the approval of our institutional ethics committee.

Conventional diabetic and normal rat models were used for an excisonal wound healing study. Two circular wounds are created on each animal, one was treated with a commercially available wound healing product, REGRANEX, and the other with 5% phenyloin sodium gel of Example 1. Regranex is a preparation containing becaplermin or human platelet derived growth factor. It was found that there was no significant difference in the time to healing for diabetic animals (p=0.29).

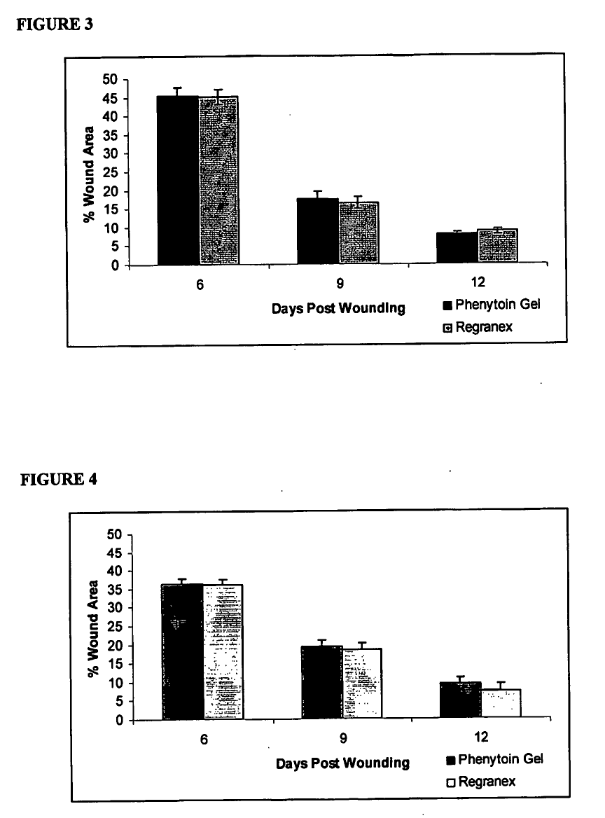

The percentage of wound area remaining for those animals treated daily with phenyloin gel equated with that for those treated with Regranex (FIG. 3). Also, there was no significant difference between the wound areas for the normal rat model (p>0.05 for all days, FIG. 4).

### Example 6

### Controlled Release Properties

The release of phenyloin sodium from the gels into a basic medium was investigated over a 24 hour period.

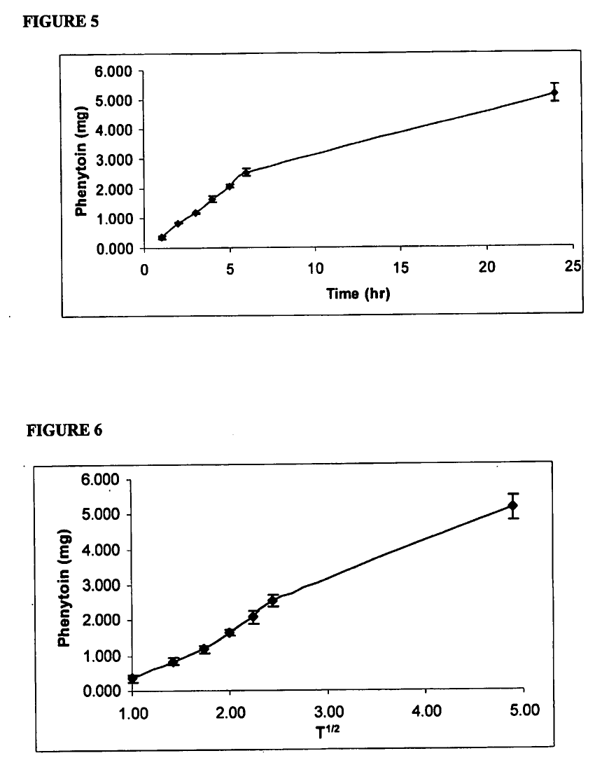

FIG. 5 illustrates the release profile of phenyloin sodium from a 5% phenyloin sodium 1% carbomer gel over a 24 hour period.

FIG. 6 illustrates the release profile of phenyloin sodium from a 5% phenyloin sodium 1% carbomer gel over a 24 hour period plotting drug released versus the root of time.

9% of the drug present in the gel applied was released over the 24 hour period.

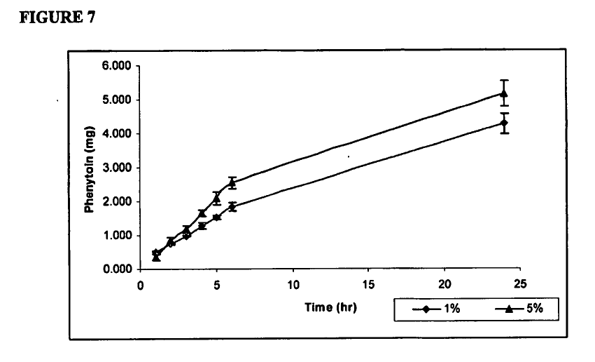

FIG. 7 provides a comparison of the release profile of phenyloin sodium from 5% phenyloin sodium and 1% phenyloin sodium gels over a 24 hour period.

The rate of release of the drug seems to be independent of the drug concentration after the first six hours, possibly showing the rate of transfer of the drug from the suspended reservoir to the soluble phase becomes constant.

### Example 7

### o/w Emulsion (Cream) Formulation

An oil in water phenyloin formulation was prepared using the following components: Phenyloin Sodium 5%; Emulsifying wax 9%; White soft paraffin 15%; Liquid paraffin 6%; Phenoxyethanol 1%; and Water, to 100%. The phenyloin sodium was first mixed into the oil phase, the stabilising agent added and then the water added slowly during vigorous mixing along with the preservative antioxidant.

### Example 8

### Phenyloin Foam Formulation

A topical phenyloin-containing formulation is prepared using the following components:

Propylene glycol; Emulsifying Wax; Polyoxyl (10) stearyl ether; Cetyl alcohol; Methyl parahydroxybenzoate; Propyl parahydroxybenzoate; Trolamine; Purified water; Hydrocarbon propellant HP-70 (consisting of isobutane and propane); and phenyloin sodium.

The phenyloin is dissolved/suspended in a mixture of the above components and stored in a pressurised aluminium container.

### Example 9

### Phenyloin Spray

A physical mixture of phenyloin sodium and a β-cyclodextrin is prepared by mixing in geometric proportions. A solid dispersion is then prepared by kneading and co evaporation. The kneaded dispersions are prepared by grinding the drug and β-cyclodextrin together for approximately 30 minutes. The powder is then kneaded with an alcohol to get a pasty consistency and dried at 40° C. for 1 hour. The resultant mixture is then suspended in a suitable propellant (a hydrofluorocarbon) and packaged in a pressurised container.

The invention is not limited to the embodiments hereinbefore described which may be varied in construction and detail without departing from the spirit of the invention.

1. A formulation of phenyloin suitable for topical application to a wound comprising a reservoir of phenyloin entrapped within a gel, and an amount of phenyloin dissolved in an aqueous component of the gel, wherein the dissolved phenyloin is in chemical equilibrium with the phenyloin entrapped within the gel, in which the gel is formed by a gelling agent suitable for forming an ion pair with the phenyloin, wherein the formation of ion-pairs between the gelling agent and the phenyloin molecules increases the stability of the reservoir of phenyloin, wherein the formulation has a pH of from 7 to 10, and wherein the gel comprises an acidic polymer.

2. A formulation as claimed in claim 1 in which the gel is formed by a gelling agent selected from the group comprising: alginic acid; alginate derivatives; chitosan; chitosan derivatives; and a carbomer.

3. A formulation as claimed in claim 2 in which the carbomer gelling agent comprises CARBOPOL.

4. A formulation as claimed in claim 1 in which the gel includes a complexing agent which physically entraps the phenyloin in the reservoir.

5. A formulation as claimed in claim 4 in which the complexing agent is selected from the group comprising: cyclodextrins; buffer salts; amino acids; small peptides; polyarginines; polyglycines; polylysines; and glutamic acid.

6. A formulation as claimed in claim 1 in which the gelling agent is present in an amount of between 0.5% and 5.0% (w/w).

7. A formulation as claimed in claim 1 in which the phenyloin is present in the formulation at an amount of between 0.5% and 10.0% (w/w).

8. A formulation as claimed in claim 1 in which the phenyloin comprises a phenyloin salt.

9. A formulation as claimed in claim 8 in which the phenyloin salt consists of sodium phenyloin.

10. A formulation as claimed in claim 1 further including one or more therapeutic molecules for the treatment of wounds.

11. A formulation as claimed in claim 10 in which the therapeutic molecules are anti-infective agents selected from the group comprising antibiotics, antifungal agents, and zinc salts.

12. A formulation as claimed in claim 1, comprising:
from 0.5% to 10.0% phenyloin salt;
from 0.5% to 5.0% gelling agent;
an aqueous base; and optionally
an amount of alkali or acid or buffer salts sufficient to adjust the pH of the formulation to between 7 and 10,
wherein the gelling agent comprises an acidic gel-forming polymer capable of forming an ion pair with the phenyloin.

13. A formulation as claimed in claim 12 in which the gelling agent is a carbomer.

14. A solid support forming part of a bandage or a dressing for wounds, wherein the solid support carries a formulation according to claim 1.

15. A solid support as claimed in claim 14 and comprising a mesh formed of woven or non-woven material.

16. A bandage or dressing for wounds comprising a solid support as claimed in claim 14.

17. A formulation as claimed in claim 1 for use as a medicament.

18. A formulation as claimed in claim 1 for use in the topical treatment of wounds.

19. A formulation as claimed in claim 1 for use in the topical treatment of wounds in diabetic patients.

20. A method of treating a wound in an individual comprising a step of administering a formulation of claim 1 topically to the wound.

21. A method of treating a wound in a diabetic patient comprising a step of administering a formulation of claim 1 topically to the wound.
